(19) United States
(12) Patent Application Publication
(10) Pub. No.: US 2026/0348954 A1
(43) Pub. Date: Nov. 5, 2026

(54) METHODS OF CHARACTERIZING NON-GLYCOSYLATED MONOCLONAL ANTIBODY AGGREGATES

(71) Applicant: Regeneron Pharmaceuticals, Inc., Tarrytown, NY (US)

(72) Inventors: Yuetian Yan, Chappaqua, NY (US); Shunhai Wang, Scarsdale, NY (US)

(21) Appl. No.: 19/663,721

(22) Filed: Apr. 30, 2026

Related U.S. Application Data
(60) Provisional application No. 63/797,504, filed on Apr. 30, 2025.
(60) Provisional application No. 63/900,341, filed on Oct. 16, 2025.

(51) Int. Cl. G01N 33/563; G01N 33/68

(52) U.S. Cl. CPC G01N 33/563; G01N 33/6848

(57) ABSTRACT

The inventions provide systems for and methods of characterizing aggregates in a monoclonal antibody preparation, wherein the method comprises the steps of (a) subjecting the monoclonal antibody preparation to a stress; (b) conducting post-column denaturation-assisted size-exclusion chromatography and mass spectrometry (SEC-PCD-MS) on the stressed monoclonal antibody preparation from step (a); and (c) analyzing elution peaks to characterize the aggregates.

### CROSS-REFERENCE TO RELATED APPLICATIONS

This application claims the benefit of U.S. Provisional Application No. 63/900,341, filed Oct. 16, 2025, and U.S. Provisional Application No. 63/797,504, filed on Apr. 30, 2025, which are each herein incorporated by reference.

### FIELD OF THE INVENTIONS

The inventions provide methods of characterizing aggregates in monoclonal antibody (mAb) preparations to facilitate improved development and production methodologies and improved antibody products. Systems for performing the methods also are provided.

### BACKGROUND

In the past decades, monoclonal antibody-based therapeutics have revolutionized the treatment landscape for a variety of human diseases, ranging from viral infections, cardiovascular diseases, autoimmune diseases, to many types of cancer. Therapeutic monoclonal antibodies are inherently complex molecules that can host a wide variety of post-translational modifications (PTMs), which may arise during their production and storage. Therefore, unlike small molecule counterparts, therapeutic mAbs are essentially a mixture of closely related proteoforms that collectively contribute to their overall properties and the therapeutic functions. Among various PTMs, the conserved N-linked glycosylation, located within the CH₂ domain of each heavy chain in all IgG subclasses, is considered a critical quality attribute that can exhibit a large degree of macro- and micro-heterogeneities during production. The presence or absence of this N-glycan, along with specific N-glycan structures, is known to impact the function and stability of the molecule. For example, IgG1 molecules lacking core fucosylation are well recognized for their improved binding to Fc gamma receptor Illa, making them highly attractive in therapeutic applications where enhanced effector function is desired. On the other hand, mAb molecules with high mannose N-glycans have been shown to exhibit suboptimal pharmacokinetic properties compared to those with complex N-glycans, showing increased clearance from the circulation and reduced half-life. In addition to therapeutic functions, the Fc N-glycosylation status can also have a significant impact on a mAb molecule's stability. In particular, the nonglycosylated mAbs (that is, produced by either PNGase F-mediated deglycosylation or glycosite mutations) have demonstrated decreased thermal stability compared to their fully glycosylated counterparts, as evidenced by reduced melting temperature reported by differential scanning calorimetry (DSC) experiments. These findings also correlated well with structural studies, such as those using hydrogen-deuterium exchange mass spectrometry (HDX-MS), suggesting that the absence of Fc N-glycan results in an increased protein backbone flexibility in the CH₂ domain. While prior reports have shown the compromised thermal stability of non-glycosylated (NG) mAb molecules, it remains unclear how these non-glycosylated variants, which are often present in relatively low abundances (for example, <20%), could affect the overall stability of mAb products under thermal stress conditions relevant to their development, such as during forced degradation studies.

Forced degradation studies, particularly those performed at elevated temperatures, are integral for the development of therapeutic mAbs. Using a temperature that is notably higher than the long-term storage temperature (for example, 4 °C to 8 °C) or the manufacturing exposure temperature (for example, 25 °C), forced degradation studies can reveal the potential degradation pathways of the monoclonal antibody molecule within a short period of time (for example, within weeks). Such studies can be performed for different purposes throughout the various stages of mAb development, including developability/manufacturability assessment for candidate selection, formulation development, evaluation and development of stability-indicating analytical methods, and comparability assessment after process changes. According to a recent industry-wide survey, the majority of the respondents used a temperature range of 40 °C to 50 °C over a period of 1-4 weeks for thermal stress when designing forced degradation studies of mAbs. This temperature range is selected because it allows for sufficient degradation of mAbs within the desired timeframe without reaching their melting temperature (Tₘ), which would lead to non-representative unfolding/denaturation. While this temperature range is comfortably below the Tₘ of a fully glycosylated mAb molecule, it is notably closer to the Tₘ of its non-glycosylated variant. Given that protein aggregation is one of the major degradation pathways for mAbs under stress, it is reasonable to speculate that the non-glycosylated variant, although present in low abundance in most mAb drug products, may more readily contribute to aggregate formation compared to its fully glycosylated counterpart during forced degradation studies. This hypothesis, however, remains largely unconfirmed in the literature, presumably due to the lack of effective analytical tools that allow for detailed aggregate characterization with glycoform-specific information.

Size exclusion chromatography (SEC) with ultraviolet (UV) detection are the most frequently used techniques to monitor the aggregate formation of mAbs under thermal stress. While this technique is robust, sensitive, and stability-indicating, it does not provide detailed characterization of the aggregates. When SEC separation is coupled with multi-angle light scattering (MALS) or native mass spectrometry (MS), it can measure the molecular weights of the aggregates, allowing for the determination of the oligomerization states (for example, dimer, trimer, or greater). However, more detailed analysis often requires the fractionation of the mAb aggregates, followed by extended characterization using various biophysical tools. For instance, combining limited proteolysis and LC-MS, the dimerization interfaces involved in mAb aggregation can be revealed at the domain levels (for example, Fab-Fab, Fab-Fc, and Fc-Fc). Moreover, MS-based bottom-up analysis has also been successfully utilized to identify the covalent crosslinks that contributed to the formation of covalent mAb aggregates. While the fractionation-based strategy is highly effective, it is also very demanding in terms of material and time. Moreover, due to the additional stress posed during the fractionation process, it may also disrupt the existing aggregates or even introduce artifactual aggregates.

### SUMMARY OF THE INVENTIONS

The inventions provide methods of characterizing aggregates in a monoclonal antibody preparation, wherein the method comprises the steps of (a) subjecting the monoclonal antibody preparation to a stress; (b) conducting post-column denaturation-assisted size-exclusion chromatography and mass spectrometry (SEC-PCD-MS) on the stressed monoclonal antibody preparation from step (a); and (c) analyzing elution peaks to characterize the aggregates. The stress may be selected from thermal stress, photo stress, low pH stress, agitation stress, sheer stress or combinations thereof, for example. In a particular embodiment, the stress is thermal stress. The monoclonal antibody preparation of step (a) can be heated to greater than 40 °C. The monoclonal antibody preparation of step (a) can be heated to about 45 °C. The monoclonal antibody preparation of step (a) can be heated for up to about four weeks. The pH of the monoclonal antibody preparation can be about 5.0 or about 6.0. The monoclonal antibody preparation can comprise sucrose and/or polysorbates. The monoclonal antibody can be in a monospecific (msAb) or a bispecific antibody (bsAb) format. The monoclonal antibody can be of an IgG1 or an IgG4 subclass. Examples of such monoclonal antibodies include an antibody that binds to PD-1 (such as cemiplimab), a bispecific antibody which binds to CD3 and BCMA (such as linvoseltamab). The non-glycosylated monoclonal antibodies preferentially contribute to large aggregate formation. The very high molecular weight aggregate peak can comprise at least one multimeric form of the non-glycosylated monoclonal antibody, wherein the multimeric form is selected from the groups consisting of a dimer, trimer, tetramer, pentamer, hexamer, heptamer and larger forms.

The non-glycosylated monoclonal antibodies subjected to a stress (e.g., thermal stress) can comprise, for example, one or more of about 1.0% to about 50%; about 2% to about 50%; about 3% to about 50%; about 4% to about 50%; about 5% to about 50%; about 10% to about 50%; about 15% to about 50%, about 20% to about 50%; about 25% to about 50%; about 30% to about 50%; about 35% to about 50%; and about 40% to about 50% of a very high molecular weight (vHMW) aggregate peak. The non-glycosylated monoclonal antibodies prior to subjecting to the stress can comprise, for example, one or more of about 0.1% to about 10%; about 0.1% to about 9.5%; about 0.1% to about 9%; about 0.1% to about 8.5%; about 0.1% to about 8%; about 0.1% to about 7.5%; about 0.1% to about 7%; about 0.1% to about 6.5%; about 0.1% to about 6%; about 0.1% to about 5.5%; about 0.1% to about 5%; about 0.1% to about 4.5%; about 0.1% to about 4%; about 0.1% to about 3.5%; about 0.1% to about 3%; about 0.1% to about 2.5%; about 0.1% to about 2%; about 0.1% to about 1.5%; and about 0.1% to about 1% of the monoclonal antibody preparation.

Systems for performing the above methods also are provided. These systems can include systems for characterizing aggregates in monoclonal antibody preparations, wherein the systems comprise post-column denaturation-assisted size-exclusion chromatography and mass spectrometry (SEC-PCD-MS) to analyze the monoclonal antibody preparations to characterize at least one non-glycosylated monoclonal antibody multimeric form and at least one glycosylated monoclonal antibody multimeric form in one or more elution peaks. The multimeric forms can be selected from the group consisting of a dimer, trimer, tetramer, pentamer, hexamer, and heptamer forms. The monoclonal antibody preparations can be subjected to a stress, such as thermal stress. The systems can further comprise size exclusion chromatography coupled with ultraviolet and mass spectrometry (SEC-UV/MS) to detect multiple size variant peaks. The SEC-PCD-MS of the systems can generate extracted ion chromatograms to elucidate that non-glycosylate monoclonal antibodies are transitioned from monomers to multimers.

Steps and components of the methods and systems according to the inventions can be arranged and utilized in a manner selected by the person skilled in the art. The methods and systems according to the inventions reveal potential degradation pathways of the monoclonal antibody molecule within a short period of time (for example, within weeks). Such studies can be performed for different purposes throughout the development and production cycle, and support improvements in antibody developability/manufacturability assessment for candidate selection, formulation development, evaluation and development of stability-indicating analytical methods, and comparability assessment after process changes.

For example, the present inventions will facilitate minimization of aggregates of antibodies by reducing the level of non-glycosylated variants in antibody preparations. Additionally, the present inventions will allow for production and formulation improvements to minimize aggregates of non-glycosylated variants.

### BRIEF DESCRIPTION OF THE FIGURES

FIGS. 1A to 1E are graphs depicting the following: FIG. 1A is a graph depicting data from SEC-UV traces of mAb-A unstressed DS sample (black trace) and 45 °C, 4 weeks stressed DS sample (dash trace). FIG. 1B is a graph depicting data from a deconvoluted mass spectrum of HMW peak from 45 °C, 4 weeks stressed mAb-A sample. FIG. 1C is a graph depicting data from a deconvoluted mass spectrum of LMW1 peak from 45 °C, 4 weeks stressed mAb-A sample. FIG. 1D is a graph depicting data from a deconvoluted mass spectrum of LMW2 peak from 45 °C, 4 weeks stressed mAb-A sample. FIG. 1E is a graph depicting data from a raw mass spectrum of vHMW peak from 45 °C, 4 weeks stressed mAb-A sample.

FIGS. 2A to 2C are graphs depicting the following: FIG. 2A is a graph depicting data from native SEC-UV trace of mAb-A 45 °C, 4 weeks stressed sample highlighting the vHMW region (P2 to P6). FIG. 2B is a graph depicting data from raw mass spectra of P1-P6 detected under native conditions. FIG. 2C is a graph depicting data from Raw mass spectra of P1 to P6 detected under PCD conditions. P1: dimer, P2: trimer, P3: tetramer, P4: pentamer, P5: hexamer, and P6: heptamer and above.

FIG. 3A is a graph depicting data from SEC-UV traces of mAb-stressed at different temperatures and different time periods. FIG. 3B is a graph depicting data from deconvoluted mass spectra with PCD.

FIGS. 4A to 4C are graphs depicting SEC-PCD-MS analyses of unstressed and 45 °C, 4 weeks stressed mAb-A DS samples: FIG. 4A is SEC-UV trace. FIG. 4B depicts deconvoluted mass spectra of vHMW and monomer regions analyzed under PCD conditions; FIG. 4C depicts XICs of non-glycosylated (NG), partially glycosylated (PG), and fully glycosylated (FG) mAb-A population from SEC-PCD-MS analysis of 45 °C, 4 weeks stressed sample.

FIGS. 5A to 5E are graphs depicting SEC-PCD-UV/MS analyses of 24 mAbs stressed under 40 °C and 45 °C for 4 weeks. FIG. 5A depicts vHMW growth after the thermal stress measured by SEC-UV. FIG. 5B depicts decrease of relative NG population in the monomer species after thermal stress. FIGS. 50, 5D and 5E depicts date from three examples of mAbs stressed under 45 °C for 4 weeks as analyzed by SEC-PCD-MS showing the redistribution of NG in vHMW peak after stress. FIG. 5C tested a monospecific IgG1. FIG. 5D tested a monospecific IgG4. FIG. 5E tested a bispecific IgG4.

FIGS. 6A to 6C are graphs depicting data from SEC-PCD-MS analyses of mAb-B samples spiked with 0%, 2%, 5%, and 20% of non-glycosylated (NG) mAb-B and stressed under 45 °C for 2 weeks. FIG. 6A depicts data from SEC-UV traces of both TO and 45 °C, 2 weeks stressed samples. FIG. 6B depicts data from deconvoluted mass spectra from the monomer regions of unstressed mAb-B samples, and monomer and vHMW regions of 45 °C, 2 weeks stressed mAb-B samples. FIG. 6C depicts experimental values of relative abundances of NG species in monomer regions of unstressed mAb-B samples and monomer and vHMW regions of 45 °C, 2 weeks stressed mAb-B samples.

FIG. 7 describes the methods and formulations used in comparability studies.

FIGS. 8A to 8D concern-antibody species arising in processes P1 (FIGS. 8A and 8C) and P2 (FIGS. 8B and 8D). FIGS. 8A and 8B depict cation exchange ultra performance liquid chromatography (CEX-UPLC) and FIGS. 8C and 8D depict size exclusion chromatography-mass spectrometry (SE-UPLC) chromatograms.

FIGS. 9A to 9D depict data from CEX-UPLC (FIGS. 9A and 9B, 13.5 minute basic peak) and SE-UPLC (FIGS. 9C and 9D, 6.5 minute HMW peak) chromatograms of mAb1 at process P2.

FIGS. 10A to 10D depict graphs showing ANCOVA results of the P2 pH 5 (FIG. 10A), P1 pH 5 (FIG. 10B), P2 pH 6 (FIG. 10C) and P1 pH 6 (FIG. 10D) 13.5 min basic and 6.5 min HMW peaks.

FIGS. 11A to 11D depict data from CEX-UPLC (FIGS. 11A and 11B) and SE-UPLC (FIGS. 11C and 11D) chromatograms and plots comparing the 13.5 min basic and 6.5 min HMW peaks.

FIGS. 12A to 12C provide deconvoluted mass spectrometry data of monomers. FIG. 12A depicts chromatogram of deconvoluted MS of monomers;

FIG. 12B depicts chromatogram of deconvoluted MS of monomer peak in P2 process; and FIG. 12C depicts chromatogram of deconvoluted MS of 6.5 min HMW peak in P2 process.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired number, value, rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of approximately +/−10% or more or less depending on the ability to perform. Thus, this term encompasses values beyond those simply resulting from systematic error.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein.

### DESCRIPTION

Post-column denaturation-assisted SEC-MS method (SEC-PCD-MS) allows rapid and in-depth characterization of mAb aggregates directly from the unfractionated drug samples. By breaking down the mAb aggregates, which are often too large and heterogeneous for meaningful MS measurement, a more detailed characterization can be achieved on the released monomeric species. This technique was used to re-examine the impact of non-glycosylated mAb variant on the overall stability of mAb drugs under stress conditions encountered in forced degradation studies.

Using SEC-PCD-MS analysis, it was discovered that, upon thermal stress, the nonglycosylated variant was preferentially depleted from the monomer SEC peak and significantly enriched in the large aggregate SEC peak. This observation was more pronounced at 45 °C compared to 40 °C and it was negligible at lower temperatures. After screening 24 different mAb molecules, it was found that the preferential aggregation of non-glycosylated variant in mAb samples was very common at 45 °C, but less so at 40 °C. Finally, by spiking a non-glycosylated mAb into its glycosylated counterpart at different levels and subjecting them to 45 °C thermal stress, it was demonstrated that (1) the reduction of non-glycosylated species in monomer form, (2) the enrichment of non-glycosylated species in large aggregates, and (3) the increase of large aggregate SEC peak were all closely correlated. The inventions demonstrate for the first time that the nonglycosylated mAb variant, although present in low abundance in most mAb drug products, preferentially contributed to large aggregate formation under thermal stress conditions commonly used in forced degradation studies.

### Enrichment of Nonglycosylated Variants

During the development of mAb-A, a forced degradation study was conducted by incubating the mAb-A formulated drug substance (FDS) 50 mg/ml) sample (50 mg/ml) at 45 °C for up to 28 days. Subsequent SEC-UV/MS analysis (size exclusion chromatography coupled with ultraviolet and mass spectrometry) revealed notable increases of multiple size variant peaks compared to TO control sample, including the low molecular weight (LMW) peak, high molecular weight (HMW) peak, and the large aggregate, that is, a very high molecular weight, (vHMW peak). SEC-UV/MS can separate biomolecules by size (SEC), quantify the biomolecules (UV) and identify molecular mass of the biomolecules (MS).

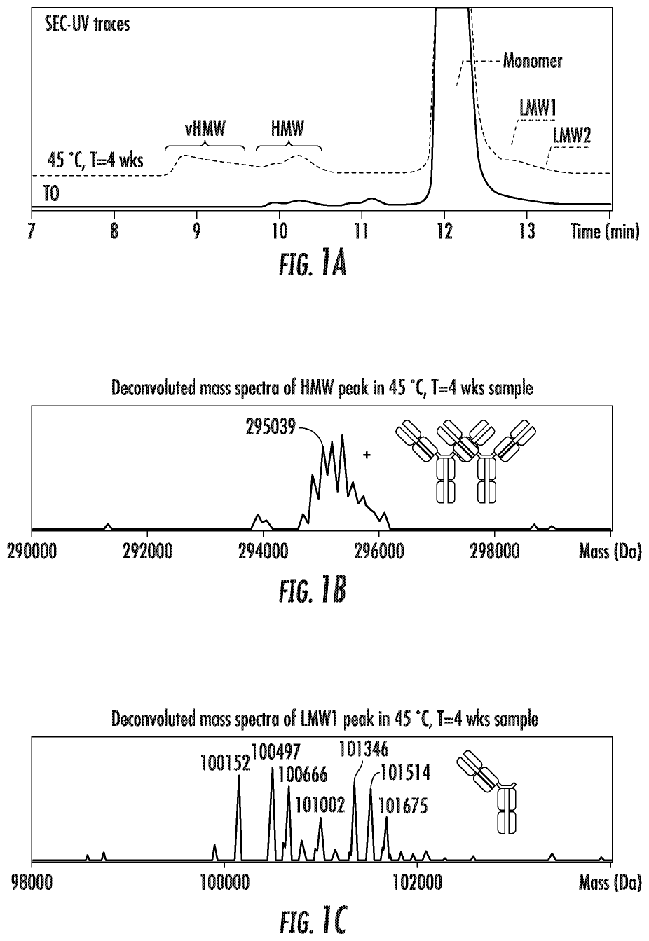

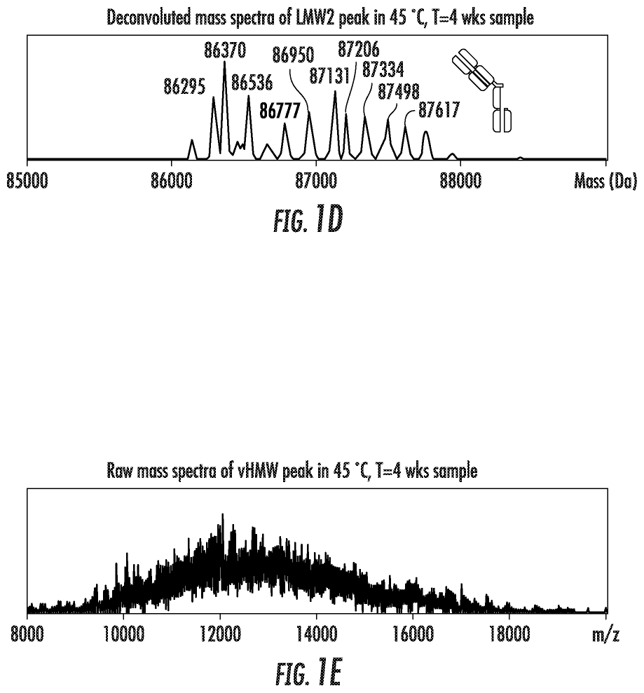

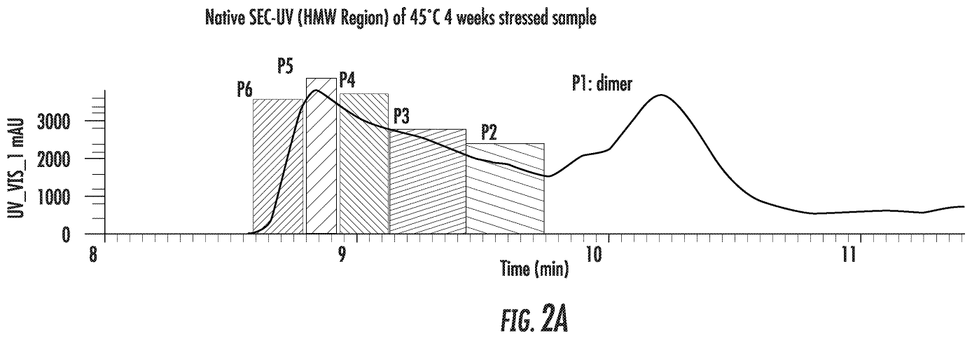

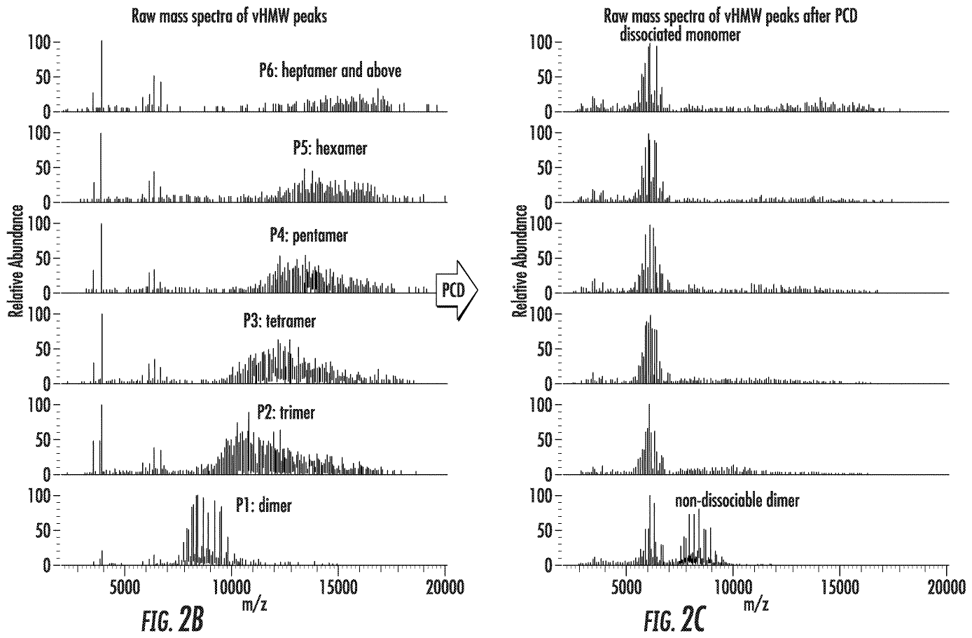

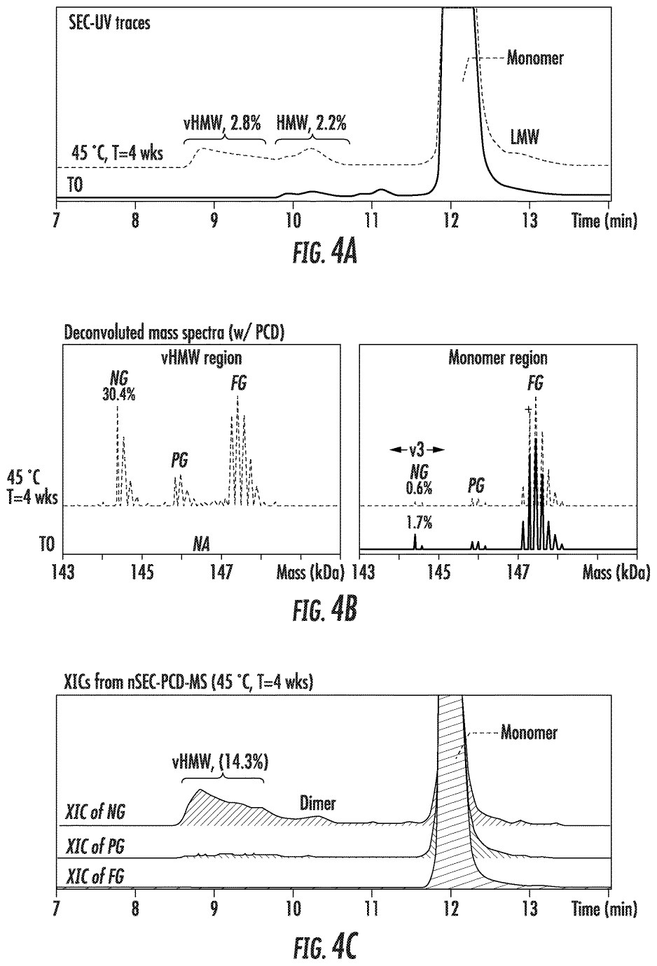

The vHMW peak showed the most significant increase compared to the other size variant peaks and was only observed upon thermal stress. Using concurrent native MS analysis, the LMW peak and HMW peak were identified as truncated mAb and mAb dimer species, respectively (FIGS. 1B and 1C). The mass spectra from the vHMW peak, on the other hand, were too heterogeneous to be deconvoluted (FIG. 1E). After comparing the m/z range from the native mass spectrum of each species within the vHMW peak with those of known mAb aggregates, it was proposed that the vHMW peak was attributed to higher order oligomers of mAb-A, such as tetramer, pentamer, hexamer, and potentially even larger species (FIGS. 2A and 2B). However, due to the large size and heterogeneity, direct MS analysis of this peak did not provide meaningful insights into its root cause. To investigate the underlying mechanism responsible for the large aggregate formation, SEC-PCD-MS analysis was performed on both the stressed sample and the TO control sample. Interestingly, under the PCD conditions (i.e., 75 mM ammonium acetate, 2% FA and 20% ACN), the large aggregates in the vHMW peak were effectively dissociated into monomers, which could be readily analyzed by MS, producing high-quality MS spectra (FIG. 2C). After deconvolution, it is clear that the mAb-A species released from the large aggregates exhibited a high level of nonglycosylated variant (30.4%) (FIG. 4B). In addition, a comparison of the deconvoluted mass spectra of the monomer SEC peak also showed a notable decrease in the non-glycosylated variant from the TO control sample (1.7%) to the thermally stressed sample (0.6%). Together, these observations suggest that the nonglycosylated variant was preferentially transitioned from the monomeric state into the large oligomeric (multimeric) state under 45 °C thermal stress. This behavior can also be readily visualized using the extracted ion chromatograms (XICs), generated from the SEC-PCD-MS analysis. Compared to the fully glycosylated main species (FIG. 4C, monomer peak), which almost entirely remained in the monomer peak, the nonglycosylated variant (FIG. 4C, XIC of NG) showed a significant enrichment in the vHMW peak and, to a much lesser extent, in the dimer peak. Interestingly, the partially glycosylated variant (FIG. 4C, XIC of PG), which has N-glycosylation on one of its two heavy chains, also showed minor enrichment in the vHMW peak. This suggests that the partially glycosylated variant also has a greater tendency to form large aggregates under thermal stress compared to the fully glycosylated main species, albeit not as much as the nonglycosylated variant. Given that the nonglycosylated antibodies are well known for their reduced thermal stability in CH2 domain, it is reasonable to hypothesize that under 45 °C thermal stress, the nonglycosylated variant may slowly unfold, exposing hydrophobic patches. These unfolding events gradually lead to the formation of large aggregates through inter-molecular hydrophobic interactions. This proposed mechanism may explain why the nonglycosylated variant was mainly enriched in the large aggregates rather than in the dimer.

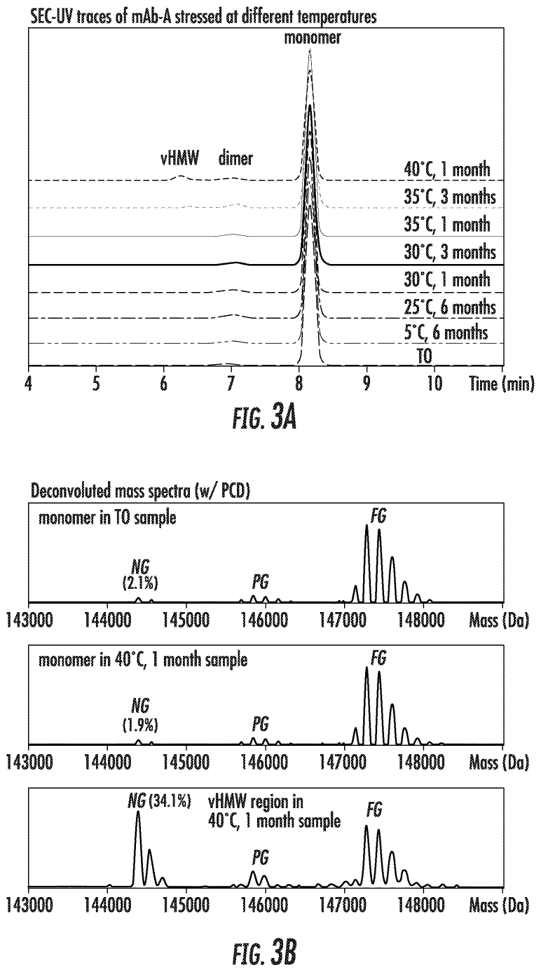

While 45 °C thermal stress is commonly applied in forced degradation studies of mAbs, other temperatures are also frequently used in various stability studies, such as long-term storage stability and accelerated stability. To examine whether the nonglycosylated variant also contributes to large aggregate formation under milder thermal stress conditions, additional thermal stability studies of mAb-A were conducted. These studies included testing at 5 °C for 6 months, 25 °C for 3 months, 30 °C for 3 months, 35 °C for 1 month, and 40 °C for 1 month. Due to material availability, a mAb-A FDS sample formulated at a higher concentration (150 mg/mL) was used. Of the conditions tested, a similar vHMW peak was only observed in the sample incubated at 40 °C for 1 month (FIG. 3A). Subsequent SEC-PCD-MS analysis of this sample also confirmed the reduction of nonglycosylated variant in the monomer peak and its corresponding enrichment in the vHMW peak (FIG. 3B). These findings indicate that a thermal stress temperature greater than 40 °C, which is close to the melting onset temperature of non-glycosylated antibodies, is likely necessary to trigger this nonglycosylation-mediated aggregation pathway. While this unique aggregation pathway is unlikely to occur under typical mAb long-term storage (4 °C to 8 °C) or accelerated stability conditions (25 °C to 37 °C), it raises important considerations when using high-temperature stability data (for example, greater than (>) 40 °C) to predict low-temperature stability through modelling approaches.

Nonglycosylation-mediated aggregation under thermal stress in diverse mAb molecules. A broad range of mAb molecules were then tested. Twenty four additional mAb molecules, encompassing both IgG1 and IgG4 subclasses, as well as monospecific (msAb) and bispecific antibody (bsAb) formats, were subjected to thermal stress under both 40 °C and 45 °C for up to 4 weeks. Prior to thermal treatment, these molecules exhibited a range of non-glycosylation levels, spanning from about 0.1% to about 5.4%, as determined by deconvoluted mass spectra (Table 1). Each molecule was examined using its respective FDS sample, with concentrations ranging from 37.1 to 217.6 mg/mL and formulation pH values spanning from 5.0 to 6.0 (Table 1). Following thermal treatment, the presence of non-glycosylated antibodies in vHMW aggregate peak was up to about 48% (Table 1). Common formulation excipients found in mAb products, such as sucrose and polysorbates, were included in most of the formulations. Therefore, Table 1 represent a diverse range of mAb FDS samples that may be encountered during development.

TABLE 1

| | | | | | | | vHMW % | | | | | NG% in |
|---|---|---|---|---|---|---|---|---|---|---|---|---|
| | Concen- | | | | | | growth | | NG % in monomer | | | vHMW |
| | tration | | | | Formulation | | 40 °C | 45 °C | | 40 °C | 45 °C | 45 °C |
| # | (mg/ml) | pl | Subclass | Type | pH | excipient | 4 wks | 4 wks | T0 | 4 wks | wks | 4 wks |
| 1 | 123.0 | 9.2 | IgG1 | msAb | 6.0 | Sucrose | 0.4% | 3.1% | 0.6% | 0.6% | 0.4% | 12.4% |
| | | | | | | Polysorbate | | | | | | |
| | | | | | | 80 | | | | | | |
| 2 | 209.7 | 9.4 | IgG1 | msAb | 6.0 | Sucrose | 0.2% | 4.7% | 0.4% | 0.4% | 0.1% | 14.8% |
| | | | | | | Polysorbate | | | | | | |
| | | | | | | 80 | | | | | | |
| 3 | 196 | 9.2 | IgG4 | msAb | 6.0 | Sucrose | 0.2% | 0.3% | 1.2% | 1.1% | 0% | 0% |
| 4 | 192.1 | 8.8 | IgG4 | msAb | 6.0 | Sucrose | 0.5% | 32.5% | 0.1% | 0.0% | 0.1% | 1.1% |
| 5 | 173.4 | 7.8 | IgG4 | msAb | 6.0 | Polysorbate | 1.5% | 50.0% | 5.4% | 4.9% | 1.7% | 3.1% |
| | | | | | | 80 | | | | | | |
| 6 | 88.8 | 9.1 | IgG4 | msAb | 6.0 | Sucrose | 0.6% | 8.0% | 5.4% | 5.1% | 4.8% | 10.4% |
| 7 | 196.4 | 8.5 | IgG4 | msAb | 5.8 | Sucrose, | 0.5% | 29.4% | 4.5% | 4.4% | 1.8% | 47.6% |
| | | | | | | Polysorbate | | | | | | |
| | | | | | | 80 | | | | | | |
| 8 | 171.1 | 9.3 | IgG4 | bsAb | 5.0 | Sucrose, | 11.6% | 7.8% | 1.3% | 0.7% | 0% | 0% |
| | | | | | | Polysorbate | | | | | | |
| | | | | | | 80 | | | | | | |
| 9 | 49.5 | 9.0 | IgG4 | bsAb | 6.0 | Polysorbate | 0.1% | 7.4% | 2.7% | 2.6% | 2.3% | 3.2% |
| | | | | | | 80 | | | | | | |
| 10 | 74.9 | 8.9 | IgG4 | msAb | 6.0 | Sucrose | 0.2% | 25.5% | 2.1% | 2.2% | 2.17% | 28.8% |
| 11 | 37.1 | 6.7 | IgG4 | msAb | 5.1 | Sucrose | 0.1% | 6.5% | 1.1% | 1.1% | 0.4% | 11.4% |
| 12 | 48.8 | 8.8 | IgG4 | msAb | 6.0 | Sucrose, | 0.2% | 6.5% | 2.3% | 2.2% | 1.8% | 24.4% |
| | | | | | | Polysorbate | | | | | | |
| | | | | | | 80 | | | | | | |
| 13 | 51.0 | 9.1 | IgG4 | msAb | 6.0 | Sucrose, | 0.2% | 5.9% | 3.0% | 2.8% | 2.7% | 36.5% |
| | | | | | | Polysorbate | | | | | | |
| | | | | | | 80 | | | | | | |
| 14 | 118.2 | 8.8 | IgG4 | bsAb | 6.0 | Sucrose, | 0.2% | 4.7% | 5.1% | 5.0% | 4.8% | 32.6% |
| | | | | | | Polysorbate | | | | | | |
| | | | | | | 80 | | | | | | |
| 15 | 78.6 | 8.6 | IgG4 | bsAb | 6.0 | Sucrose | 0.2% | 213% | 5.0% | 5.0% | 4.8% | 33.7% |
| 16 | 77.1 | 8.9 | IgG4 | bsAb | 6.0 | Sucrose | 0.2% | 3.7% | 1.8% | 1.7% | 1.3% | 12.5% |
| 17 | 150.2 | 9.0 | IgG4 | bsAb | N.A. | Sucrose | 0.7% | 24.7% | 0.1% | 0.1% | 0.0% | 43% |
| | | | | | | Polysorbate | | | | | | |
| | | | | | | 80 | | | | | | |
| 18 | 193.0 | 8.8 | IgG4 | msAb | N.A. | Sucrose | 3.2% | 5.2% | 0.6% | 0.6% | 0.2% | 2.0% |
| 19 | 79.7 | 7.5 | IgG4 | msAb | 6.0 | Sucrose | 0.7% | 10.4% | 3.3% | 3.3% | 2.6% | 22.0% |
| 20 | 190.0 | 8.9 | IgG4 | bsAb | 5.5 | Sucrose | 0.5% | 65.8% | 1.3% | 1.1% | 0.1% | 17.6% |
| 21 | 217.6 | 9.1 | IgG1 | msAb | 6.0 | Sucrose | 0.2% | 123% | 3.9% | 3.9% | 3.9% | 11.6% |
| 22 | 77.5 | 8.9 | IgG4 | bsAb | 6.0 | Sucrose | 0.2% | 2.3% | 1.8% | 1.8% | 1.5% | 16.8% |
| 23 | 49.9 | 8.3 | IgG4 | One | 6.0 | Sucrose, | 0.3% | 7.0% | 4.1% | 4.1% | 2.5% | 36.2% |
| | | | | arm | | Polysorbate | | | | | | |
| | | | | mAb | | 80 | | | | | | |
| 24 | 49.2 | 7.8 | IgG4 | msAb | 6.0 | Sucrose, | 0.1% | 1.5% | 5.1% | 5.1% | 4.8% | 42.1% |
| | | | | | | Polysorbate | | | | | | |
| | | | | | | 80 | | | | | | |

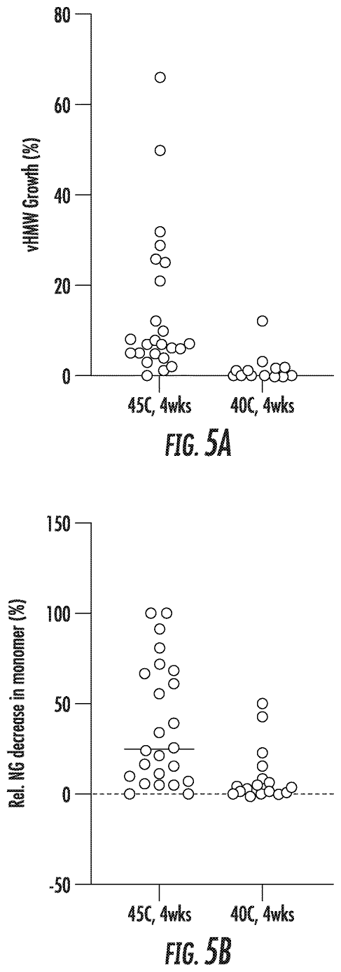

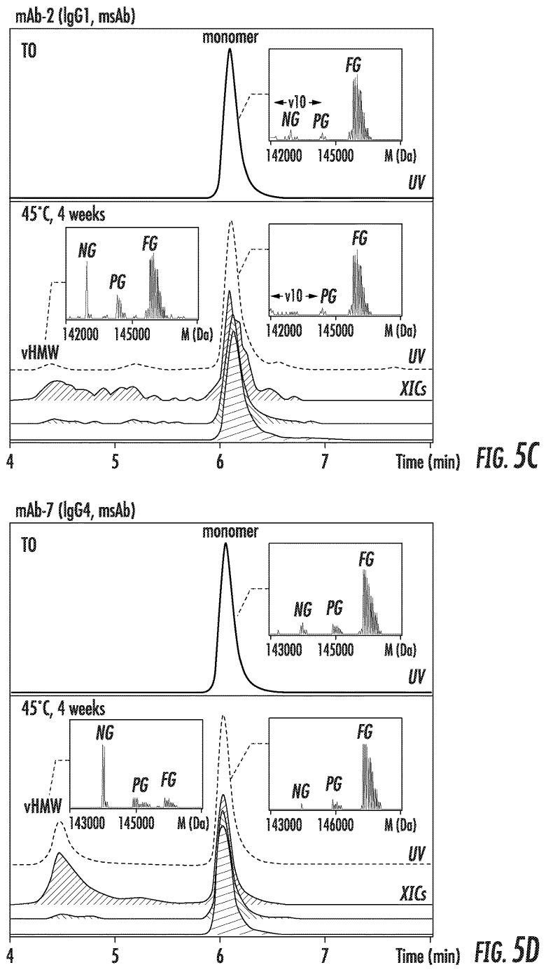

As shown in FIG. 5A, a majority of mAb molecules experienced a notable growth of large aggregates under 45 °C thermal stress, with an average increase of about 10% in vHMW peak abundance after 4 weeks. This trend and the magnitude of increase were much lower for 40 °C thermal stress. Concomitantly, a larger decrease in the relative abundance of the nonglycosylated variant in the monomer SEC peak was also observed for most mAb molecules under 45 °C stress compared to those under 40 °C stress (FIG. 5B). While most mAb molecules exhibited an anticipated enrichment of nonglycosylated variant in large aggregates under 45 °C stress, a few molecules were exceptions. In these cases, it is highly likely that the large aggregates, mediated by nonglycosylation under thermal stress, may grow to a size that can no longer be recovered by SEC. Three examples were highlighted: an IgG1 msAb (FIG. 5C), an IgG4 msAb (FIG. 5D), and an IgG4 bsAb (FIG. 5E), each demonstrating the simultaneous formation of a vHMW peak, a reduction in the nonglycosylated variant within the monomer SEC peak, and its enrichment in the vHMW SEC peak. These results demonstrate that this nonglycosylation-mediated aggregation pathway can occur in both IgG1 and IgG4 subclasses, in either monospecific or bispecific formats. Notably, mAb-2 (FIG. 5C) and mAb-20 (FIG. 5E) FDS samples only contained 0.4% and 1.3% of the non-glycosylated variant, respectively. Despite this, the formation of noticeable large aggregates was still observed under 45 °C stress. This study demonstrated that the nonglycosylation-mediated aggregation under thermal stress might be prevalent and could play an important role in the degradation pathways of a diverse range of mAb molecules in forced degradation studies during their development. Finally, as both 40 °C and 45 °C are widely utilized throughout the industry for forced degradation studies of mAbs this study offers valuable insights into the rationale of selecting the appropriate temperature for such studies and highlights important considerations for data interpretation.

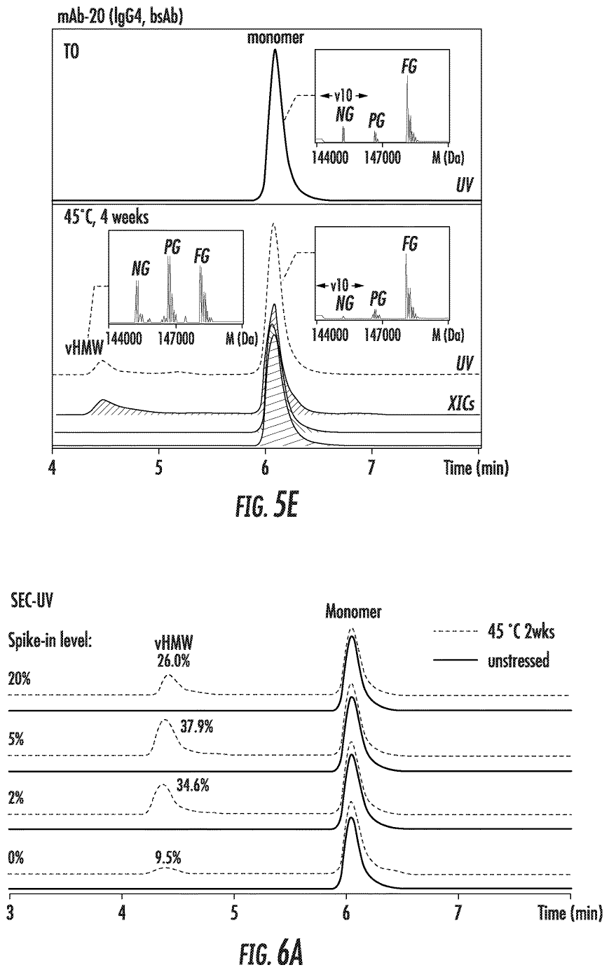

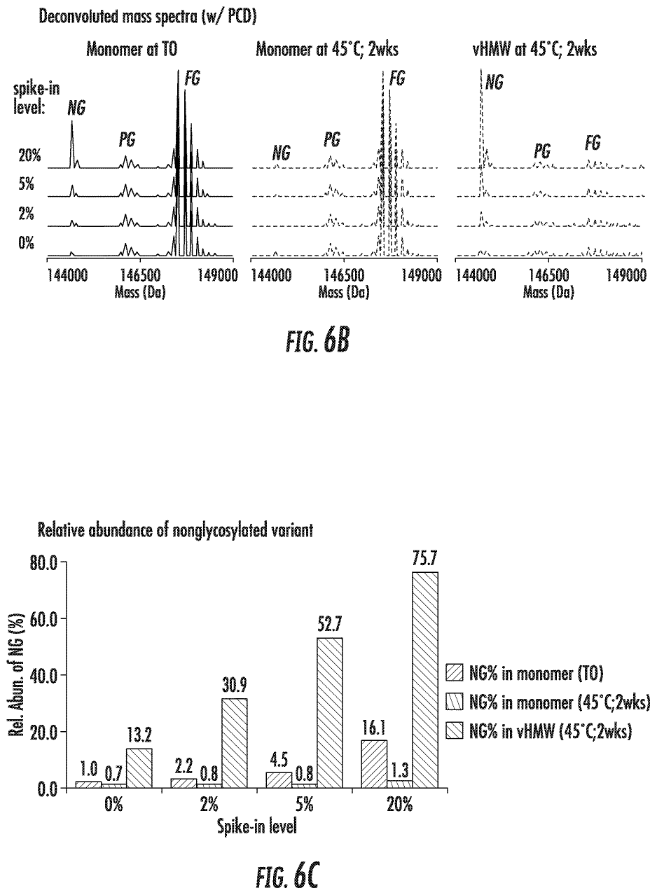

The levels of nonglycosylation correlate with large aggregation formation under thermal stress. While different mAb molecules might exhibit different tendencies to form large aggregates via the nonglycosylation-mediated pathway, it is reasonable to assume that, for the same mAb molecule, the degree of nonglycosylation should correlate with large aggregate formation. To verify this, a spike-in experiment was performed by mixing the mAb-B sample with its nonglycosylated counterpart, creating a series of samples with increasing levels of nonglycosylation. The nonglycosylated (or aglycosylated) mAb-B was produced by substituting the N-glycosite Asn297 with Gln, resulting in a homogeneous pool of mAb molecules lacking any Fc N-glycans. Samples with 4 spike-in levels, 0%, 2%, 5%, and 20%, were prepared and subsequently subjected to thermal stress at 45 °C for 2 weeks. After thermal stress, SEC-UV analysis revealed a significant increase in the relative abundances of large aggregates (i.e., vHMW), from 9.5% in the “0%” spike-in sample to 37.9% in the “5%” spike-in sample, suggesting a positive correlation between the nonglycosylation level and large aggregate formation (FIG. 6A). Interestingly, this trend was reversed for the “20%” spike-in sample, which showed a slight decrease in vHMW peak abundance compared to the “2%” and “5%” spike-in samples. It is hypothesized that the large aggregates formed in this sample may grow to a size that can no longer be recovered by SEC analysis. By examining the deconvoluted mass spectra of the monomer and vHMW SEC peaks obtained from SEC-PCD-MS analysis, it is evident that the nonglycosylated variant was preferentially transitioned from the monomeric state into large aggregates under 45 °C stress (FIG. 6B). This is particularly apparent for the “20%” spike-in sample, where the highly abundant nonglycosylated variant within the monomer peak of the TO sample dramatically reduced after 2 weeks. Concomitantly, it became the predominant component of the newly formed large aggregates. This phenomenon is consistent across all spike-in samples and is more clearly illustrated by the bar graph, summarizing the abundance changes of the nonglycosylated variant in both monomer and vHMW peaks, before and after thermal stress (FIG. 6C). Together, this study demonstrated that the level of nonglycosylation in an mAb sample correlated with the formation of large aggregation under 45 °C thermal stress, which was mediated by the nonglycosylated variant.

Nonglycosylated mAb molecules exhibit reduced thermal stability, it is still unclear how these variants, which are often present in relatively low abundances (for example, less than 20%), might impact the overall stability of mAb products when subjected to thermal stress conditions in forced degradation studies. This challenge is mainly due to the low abundance of nonglycosylated variants in a typical mAb sample, as well as the lack of effective analytical tools for detailed characterization of large mAb aggregates. The SEC-PCD-MS method was applied to re-examine the impact of nonglycosylation on mAb aggregation under thermal stress conditions. For the first time, it was determined that nonglycosylated variants directly contributed to the formation of large aggregates under thermal stresses of 45 °C and 40 °C, conditions commonly employed in forced degradation studies of mAbs. By breaking down the large mAb aggregates into monomers, SEC-PCD-MS method clearly revealed that the nonglycosylated variant was preferentially depleted from the monomeric states and was enriched within the large aggregates. This unique mechanism was absent under milder thermal stress conditions, indicating that a temperature near the melting point of nonglycosylated mAb molecules is necessary. By screening a diverse set of mAb molecules with varying properties and formulations, it was demonstrated that this nonglycosylation-mediated aggregation pathway under thermal stress may be widely present in mAb molecules. In addition, the occurrence and extent of this aggregation pathway were significantly more pronounced at 45 °C compared to 40 °C. Finally, using spike-in samples, it was demonstrated that the levels of nonglycosylation directly correlated with the formation of large aggregates under 45 °C stress, which was mediated by nonglycosylated variant. The inventions not only highlighted the complexity of the degradation pathways for mAb molecules under thermal stress but also offered valuable insights for selecting appropriate temperature for performing forced degradation studies. While 45 °C thermal stress is more likely to reveal this nonglycosylation-mediated aggregation, it may also obscure other degradation pathways that are more subtle. Finally, this finding also highlights important considerations when employing high-temperature stability data (for example, >40 °C) to predict low-temperature stability through modelling approaches, given that distinct degradation mechanisms may be involved.

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

### EXAMPLES

### Example 1—Materials and Analytical Methods for Comparative Stress Study

Stress stability was performed at 3 months incubation at 40 °C/75% relative humidity (RH) or 1 month incubation at 45 °C. Both conditions provided similar amounts of degradation for this study. All formulations in comparability study formulated to 50 mg/mL. Analytical Methods included SE-UPLC (size exclusion ultra performance liquid chromatography), CEX-UPLC (cation exchange ultra performance liquid chromatography), LC-MS (liquid chromatography-mass spectrometry), and SEC-MS (size exclusion chromatography-mass spectrometry).

Materials. Deionized water was provided by a Milli-Q integral water purification system installed with a MilliPak Express 20 filter (Millipore Sigma, Burlington, MA). Invitrogen UltraPure 1M Tris-HCl buffer, pH 7.5, and acetonitrile (ACN) (LC-MS grade) were purchased from Thermo Fisher Scientific (Waltham, MA). Formic acid (Fluka) was purchased from Honeywell (Morris Plains, NJ). Ammonium acetate (LC-MS grade) and 2-Propanol (IPA; HPLC grade) were purchased from Sigma Aldrich (St. Louis, MO). All therapeutic mAbs used in this study were produced in-house at Regeneron (Tarrytown, NY).

SEC-PCD-MS Analysis. PCD-assisted SEC-MS was performed as described previously. Briefly, Native size exclusion chromatography (nSEC) was performed on an UltiMate 3000 UHPLC System (Thermo Fisher Scientific, Bremen, Germany) using an Acquity BEH200 SEC column (4.6×150 mm, 200 Å, 1.7 μm, Waters, Milford, MA) with the column temperature of 30 °C. The antibody size variants were eluted by an isocratic flow of 150 mM ammonium acetate at 0.2 mL/min. For post-column denaturation (PCD), a denaturing solution consisting of 60% ACN, 36% water, and 4% FA was delivered by a secondary pump at a flow rate of 0.2 mL/min and mixed with the SEC eluent (1:1 ratio) using a T-mixer before MS detection. To allow for online native MS analysis, the combined analytical flow (0.4 mL/min) was split into a microflow (<10 μL/min) for nanoelectrospray ionization (NSI)-MS detection and the remaining high flow for UV detection. A Thermo Q Exactive UHMR (Thermo Fisher Scientific, Bremen, Germany) equipped with a microflow-nanospray electrospray ionization (MnESI) source and a microfabricated monolithic multinozzle (M3) emitter (Newomics, Berkeley, CA) was used for native MS analysis.

Data Analysis. Intact mass spectra from nSEC-MS analysis under native or PCD conditions were deconvoluted using Intact Mass software from Protein Metrics (Cupertino, CA). Extracted ion chromatograms were generated using XCalibur from Thermo Fisher Scientific (Bremen, Germany). Statistical Analysis used GraphPad Prism, Pearson correlation test and Analysis of Covariance (ANCOVA).

### Example 2—Comparative Studies

As explained above, SEC-PCD-MS analysis demonstrated that upon thermal stress, the nonglycosylated variant was preferentially depleted from the monomer SEC peak and significantly enriched in the large aggregate SEC peak. This observation was more pronounced at 45 °C compared to 40 °C and it was negligible at lower temperatures. The data provided herein further confirm the improvements provided by the inventions.

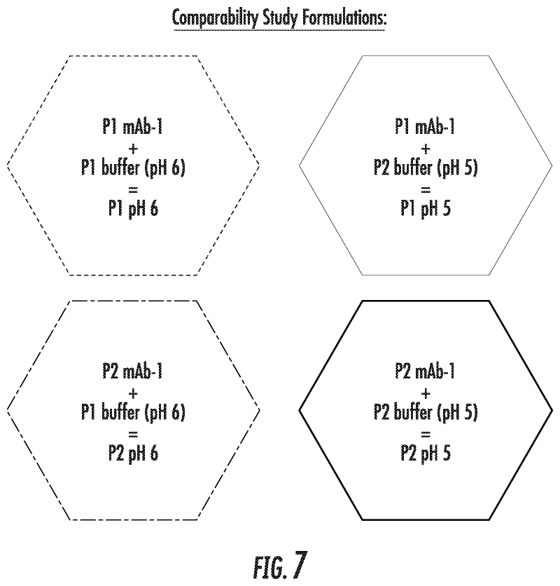

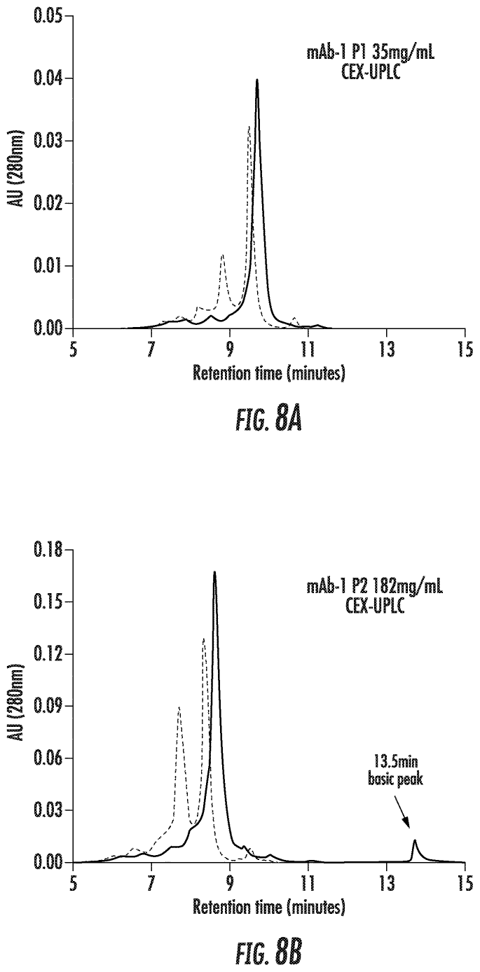

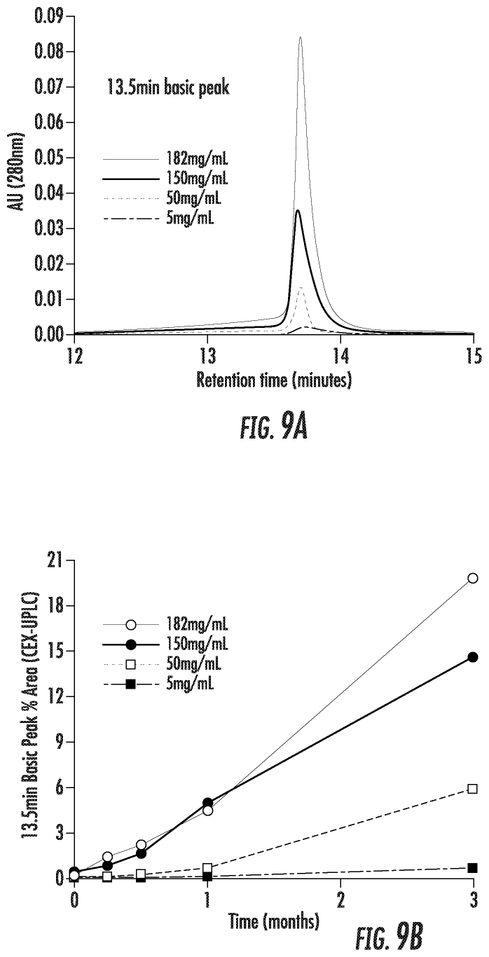

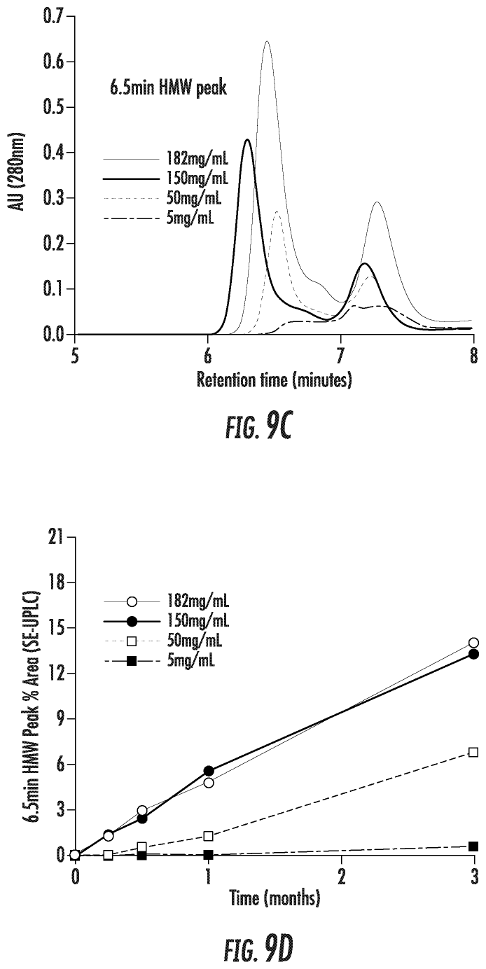

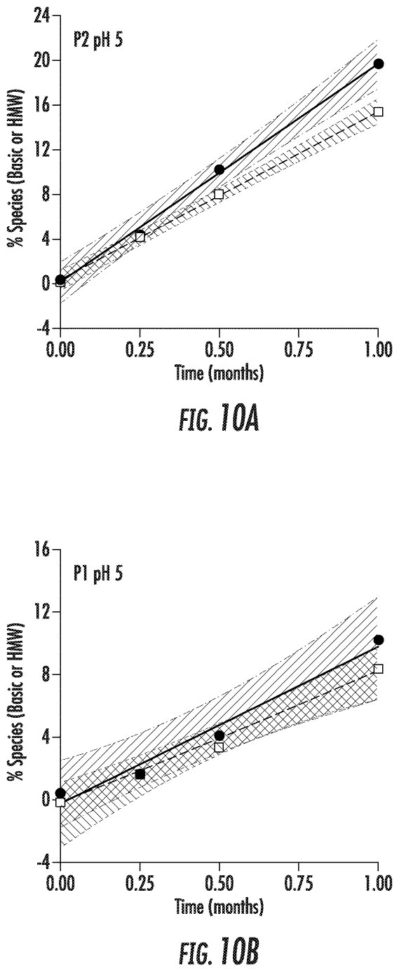

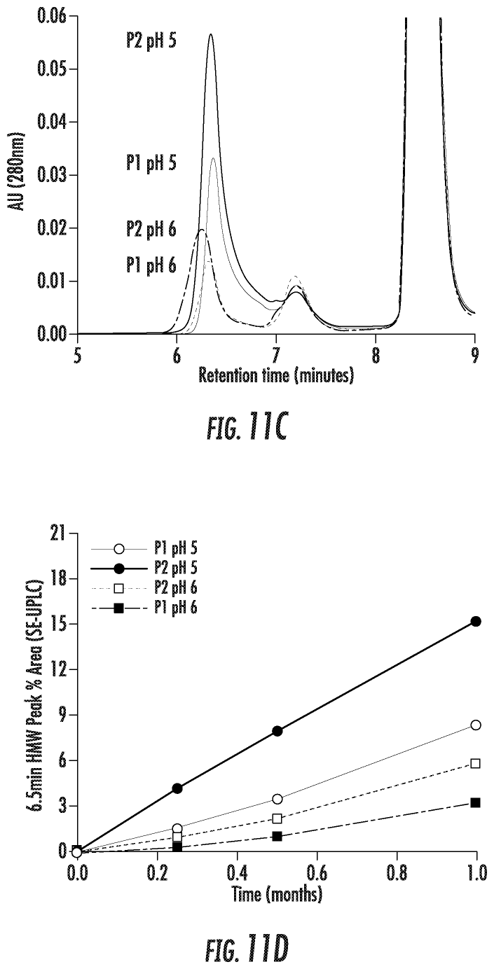

A novel species was observed following a process change (P1 to P2). Monoclonal antibody 1 (mAb-1) was further analyzed in an early-stage (P1) and a late-stage (P2). At the P1 stage, smaller batch sizes of mAb-1 for Phase I/IIa clinical trials and are lyophilized to maximize stability. A histidine buffer at pH 6.0 buffer, and typically at a low protein concentration of 2 to 35 mg/mL. FIG. 7 sets forth formulations used in comparability studies of FIGS. 9A-9D and 11A-11D.

At the P2 stage, larger batch sizes are needed for Larger batch sizes for Phase Ilb/Ill clinical trials, and are in a more molecule-specific liquid formulation comprising acetate buffer at pH 5.0. The protein concentration is higher, typically 5 to ≥180 mg/mL.

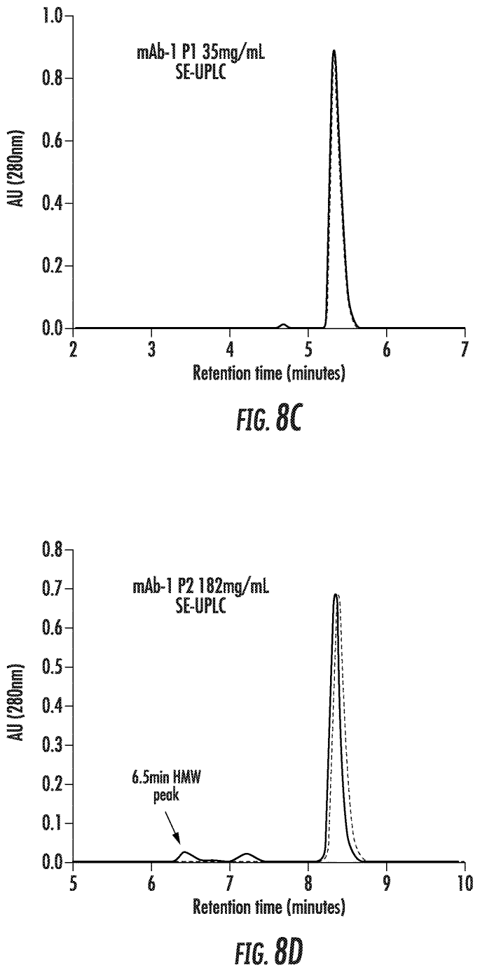

As depicted in FIGS. 8A-8D, a basic charge variant (referred to as 13.5 min basic peak) was observed by cation exchange-ultra-performance liquid chromatography (CEX-UPLC) in thermal stressed mAb-1 manufactured using the late-stage process (P2 in FIGS. 8B and 8D) that was not observed in mAb-1 manufactured from the early-stage process (P1 in FIGS. 8A and 8C). Additionally, a larger than dimer HMW peak (referred to as 6.5 min HMW peak) emerged in P2 mAb-1 following thermal stress by size exclusion-UPLC (SE-UPLC) (FIG. 8D, that was not observed in P1 mAb-1 (FIG. 8C).

FIGS. 9A-9D depict data from CEX-UPLC (FIGS. 9A-9B) and SE-UPLC (FIGS. 9C-9D) chromatograms of P2F2 mAb1 showing the 13.5 min basic and 6.5 min HMW peaks, respectively, at the end of 3 months incubation at 40 °C/75% RH, with the same column load. The growth of the 13.5 min basic peak and 6.5 min HMW peak of P2 mAb-1 in the four protein concentrations tested were plotted through stress stability (3 months incubation at 40 °C/75% RH) to determine the growth rates of these species. Pearson correlation coefficient shows statistically significant correlation between the 13.5 min basic peak and the 6.5 min HMW peak growth rates at each protein concentration (see Table 2). Accordingly, formation of the 13.5 min basic and 6.5 min HMW peaks in mAb-1 P2 are protein concentration dependent.

TABLE 2

| Formulation | | | | |
|---|---|---|---|---|
| (P2) | 182 mg/mL | 150 mg/mL | 50 mg/mL | 5 mg/mL |
| Person r | 0.9912 | 0.9936 | 0.9968 | 0.9959 |
| R² | 0.9825 | 0.9873 | 0.9936 | 0.9918 |
| Significant? | Yes | Yes | Yes | Yes |
| _{P} value | 0.0010 | 0.0006 | 0.0002 | 0.0041 |

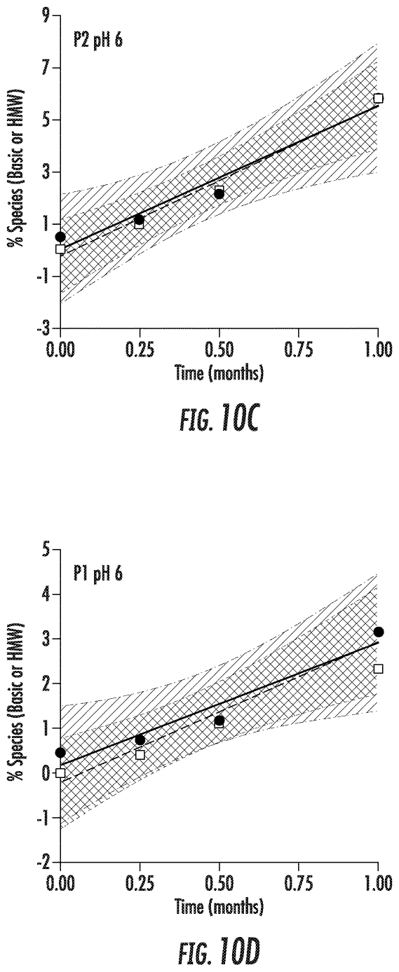

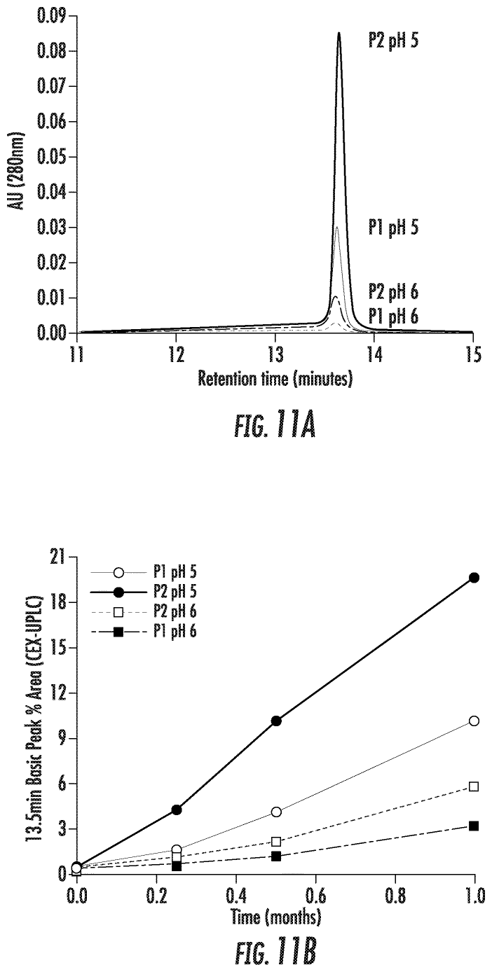

FIGS. 10A-10D depict graphs showing ANCOVA results of the P2 pH 5 (FIG. 10A), P1 pH 5 (FIG. 10B), P2 pH 6 (FIG. 10C) and P1 pH 6 (FIG. 10D) 13.5 min basic and 6.5 min HMW peaks, and shows statistical difference in their slopes. The P2 pH 5 (FIG. 10A) sample has slopes that begin to diverge around 0.75 months. This divergence is likely due to the formation of a larger than dimer species that begins to show as a shoulder on the right slope on the 6.5 min HMW peak.

ANCOVA results of the P1 pH 5 (FIG. 10B), P2 pH 6 (FIG. 10C), and P1 pH 6 (FIG. 10D) 13.5 min basic and 6.5 min HMW peaks show no statistical difference in their slopes. Indicating correlation between the two peaks

In FIGS. 10A-10D, the 13.5 min basic peak is in solid black circle and the 6.5 min HMW peak is square. Time points are shown in solid circles and clear squares; linear regression fits of time points are lines and the 95% confidence interval of the linear regression is shown as a filled-in area bracketed by dotted lines.

FIGS. 11A-11D depict data from CEX-UPLC (FIGS. 11A-11B) and SE-UPLC (FIGS. 11C-11D) chromatograms and plots comparing the 13.5 min basic and 6.5 min HMW peaks in the comparability formulations displayed increased growth rates at lower pH. Pearson correlation (see Table 3) demonstrated significant correlation between the growth rates of the 13.5 min basic and 6.5 min HMW peaks in each of the four formulations, with the greatest intensity as follows: P2 pH 5>P1 pH 5>P2 pH 6>P1 pH 6. Lower pH contributes to increased amounts of the two peaks, but within each pH, P2 has greater amounts than P1. Within the same pH, P2 consistently has more of each peak than P1.

TABLE 3

| Formulation | P2 pH 5 | P1 pH 5 | P2 pH 6 | P1 pH 6 |
|---|---|---|---|---|
| Pearson r | 0.9972 | 0.9980 | 0.9967 | 0.9958 |
| R² | 0.9944 | 0.9960 | 0.9934 | 0.9916 |
| Significant? | Yes | Yes | Yes | Yes |
| p value | 0.0028 | 0.0020 | 0.0033 | 0.0042 |

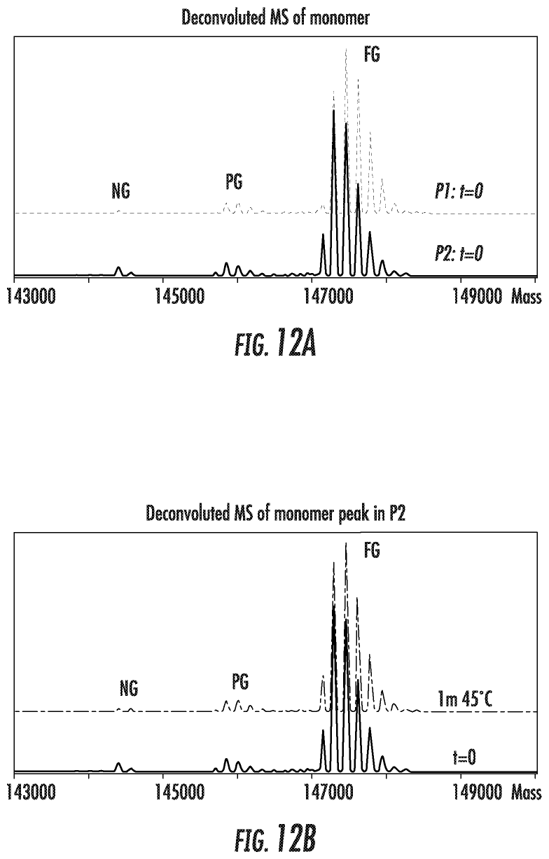

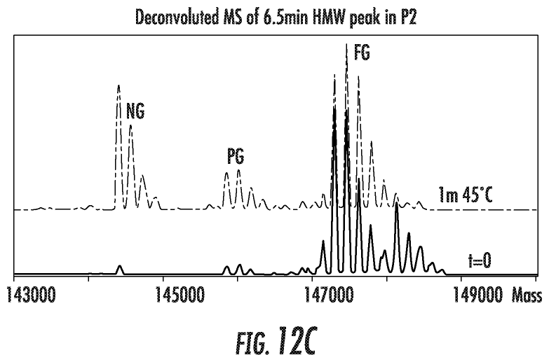

FIGS. 12A-12C provide deconvoluted mass spectrometry data of monomers. Unstressed P1 pH 6 and P2 pH 5 samples from the comparability study were analyzed via MS to determine the base levels of glycosylated species present in each (Top). While both P1 pH 6 and P2 pH 5 samples contain fully glycosylated (FG), partially glycosylated (PG) and non-glycosylated (NG) species, P2 pH 5 contains higher levels of NG species when compared to P1 pH.

SEC-MS results indicate that non-glycosylated species were preferentially depleted from the mAb-1 P2 monomer population (middle left) and enriched in the 6.5 min HMW peak (middle right) following thermal stress. After thermal stress, the ratio of NG/FG was found to be decreased in the monomer fraction, elevated in the dimer fraction and significantly elevated in the 6.5 min HMW species when analyzed by SEC-MS (see Table 4). The data demonstrate that mAb from P2 process contains a higher level of non-glycosylated species which preferentially aggregate following thermal stress.

TABLE 4

| | Non-glycosylated species/ | | |
|---|---|---|---|
| P2 mAb-1 | fully glycosylated species | | |
| samples | Monomer | Dimer | 6.5 min HMW |
| Unstressed | 0.0254 | 0.0026 | Not detectable |
| Stressed | 0.002 | 0.049 | 0.311 |
| (45° C. 1 m) | | | |

No other new species were identified in mAb-1 P2 material. The 13.5 min basic species was first observable in the P2 material due to a combination of lower pH, higher protein concentration, and process. The 13.5 min basic species is only observable in P1 when protein concentration is increased to ≥50 mg/mL and then subjected to thermal stress. P2 mAb-1 has a higher amount of non-glycosylated species, which are prone to form HMW species and are preferentially depleted from the monomer species and enriched in the 6.5 min HMW species following thermal stress. The data collectively demonstrate that increased levels of non-glycosylated species from the mAb1 P2 can be the reason why elevated HMW species increase regardless of formulation.

It is to be understood that the description, specific examples, and data, while indicating exemplary embodiments, are given by way of illustration, and are not intended to limit the inventions. Various changes and modifications within the inventions, including combining embodiments in whole and in part, will become apparent to the skilled artisan from the discussion, disclosure and data contained herein, and thus are considered part of the inventions.

What is claimed is:

1. A method of characterizing aggregates in a monoclonal antibody preparation, wherein the method comprises the steps of
(a) subjecting the monoclonal antibody preparation to a stress;
(b) conducting post-column denaturation-assisted size-exclusion chromatography and mass spectrometry (SEC-PCD-MS) on the stressed monoclonal antibody preparation from step (a); and
(c) analyzing elution peaks to characterize the aggregates.

2. The method according to claim 1, wherein the monoclonal antibody preparation of step (a) is subjected to a thermal stress.

3. The method according to claim 1, wherein the monoclonal antibody preparation of step (a) is heated to about 40 °C or above.

4. The method according to claim 2, wherein the monoclonal antibody preparation of step (a) is heated for up to about four weeks.

5. The method according to claim 1, wherein pH of the monoclonal antibody preparation is about 5.0 or about 6.0.

6. The method according to claim 1, wherein the monoclonal antibody preparation comprises sucrose and/or polysorbates.

7. The method according to claim 1, wherein the monoclonal antibody is a monospecific antibody or a bispecific antibody.

8. The method according to claim 7, wherein the monoclonal antibody is a monospecific antibody.

9. The method according to claim 7, wherein the monoclonal antibody is a bispecific antibody.

10. The method according to claim 1, wherein non-glycosylated monoclonal antibodies form a portion of the aggregate.

11. The method according to claim 10, wherein the non-glycosylated monoclonal antibodies subjected to the stress comprise about 1.0% to about 50% of a very high molecular weight aggregate peak.

12. The method according to claim 11, wherein the non-glycosylated monoclonal antibodies prior to subjecting to the stress comprise about 0.1% to about 10% of the monoclonal antibody preparation.

13. The method according to claim 11, wherein non-glycosylated monoclonal antibodies preferentially contribute to large aggregate formation.

14. The method according to claim 11, wherein the very high molecular weight aggregate peak comprises at least one multimeric form of the non-glycosylated monoclonal antibodies, wherein the multimeric form is selected from the group consisting of a dimer, trimer, tetramer, pentamer, hexamer, and heptamer forms.

15. A system for characterizing aggregates in a monoclonal antibody preparation, wherein the system comprises post-column denaturation-assisted size-exclusion chromatography and mass spectrometry (SEC-PCD-MS) to analyze the monoclonal antibody preparation to characterize at least one non-glycosylated monoclonal antibody multimeric form and at least one glycosylated monoclonal antibody multimeric form in one or more elution peaks.

16. The system according to claim 15, wherein the multimeric form is selected from the group consisting of a dimer, trimer, tetramer, pentamer, hexamer, and heptamer forms.

17. The system according to claim 15, wherein the monoclonal antibody preparation is subjected to a stress.

18. The system according to claim 15, further comprising size exclusion chromatography coupled with ultraviolet and mass spectrometry (SEC-UV/MS) to detect multiple size variant peaks.

19. The system according to claim 15, wherein the SEC-PCD-MS can generate extracted ion chromatograms to elucidate that non-glycosylate monoclonal antibodies are transitioned from monomers to multimers.

20. The system according to claim 15, further comprising size exclusion chromatography coupled with ultraviolet and mass spectrometry (SEC-UV/MS) to detect multiple size variant peaks and wherein he SEC-PCD-MS can generate extracted ion chromatograms to elucidate that non-glycosylate monoclonal antibodies are transitioned from monomers to multimers.
